(19) United States
(12) Patent Application Publication
(10) Pub. No.: US 2021/0322309 A1
(43) Pub. Date: Oct. 21, 2021

(54) Inhalable Formulation of a Solution Containing Olodaterol

(71) Applicants: Cai Gu Huang, Shrewsbury, MA (US); Pengpeng Gu, Shanghai (CN)

(72) Inventors: Cai Gu Huang, Shrewsbury, MA (US); Pengpeng Gu, Shanghai (CN)

(73) Assignee: Cai Gu Huang, Shrewsbury, MA (US)

(21) Appl. No.: 17/231,727

(22) Filed: Apr. 15, 2021

Related U.S. Application Data
(60) Provisional application No. 63/011,220, filed on Apr. 16, 2020.

(51) Int. Cl. A61K 9/00; A61K 47/10; A61K 47/14; A61K 47/18; A61K 47/12; A61K 31/538; A61K 47/02; A61K 9/08

(52) U.S. Cl. CPC A61K 9/0078; A61K 47/10; A61K 47/14; A61K 9/08; A61K 47/12; A61K 31/538; A61K 47/02; A61K 47/186

(57) ABSTRACT

The present invention relates to a liquid, propellant-free pharmaceutical preparation and a method for administering the pharmaceutical preparation by nebulizing the pharmaceutical preparation in an inhaler. The propellant-free pharmaceutical preparation comprises: (a) Olodaterol; (b) a solvent; (c) an isoosmotic adjusting agent; (d) a pH adjusting agent, and optionally including other pharmacologically acceptable additives.

### PRIORITY STATEMENT

This application claims the benefit of the filing date of U.S. Provisional Patent Application No. 63/011,220, filed on Apr. 16, 2020, the contents of which are incorporated herein by reference in its entirety.

### BACKGROUND OF THE INVENTION

Olodaterol hydrochloride, is chemically described as 2H-1,4-Benzoxazin-3H(4H)-one, 6-hydroxy-8-[(1R)-1-hydroxy-2-[[2-(4-methoxyphenyl)-1,1-dimethylethyl]-amino]ethyl]-, monohydrochloride, is disclosed in U.S. Pat. Nos. 7,220,742, 7,491,719, 7,056,916, 7,727,984, and has the following chemical structure:

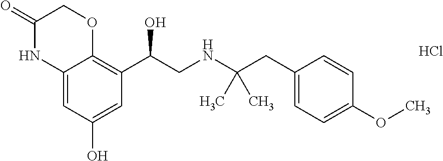 [SMILES: COc1ccc(CC(C)(C)NC[C@H](O)c2cc(O)cc3c2OCC(=O)N3)cc1 ; Cl]

Olodaterol is a long-acting beta2-adrenergic agonist (LABA) that activates beta-2 adrenoreceptors on airway smooth muscle, causing bronchodilation. Beta-2 receptors are the adrenergic receptors in bronchial smooth muscle. Olodaterol can provide therapeutic benefit in the treatment of asthma or chronic obstructive pulmonary disease, including chronic bronchitis and emphysema.

The present invention relates to a propellant-free inhalable formulation of Olodaterol, or a pharmaceutically acceptable salt or solvate, dissolved in water, in conjunction with inactive ingredients preferably administered using a nebulization inhalation device, and propellant-free inhalable aerosols resulting therefrom. The pharmaceutical formulations disclosed in the current invention are especially suitable for nebulization inhalation, which have better lung depositions (typically up to 55-60%), as compared to dry powder inhalation formulations.

The pharmaceutical formulations of the present invention are particularly suitable for administering the active substances by nebulization inhalation, especially for treating asthma and chronic obstructive pulmonary disease.

### SUMMARY OF THE INVENTION

The present invention relates to pharmaceutical formulations of Olodaterol and its pharmaceutically acceptable salts or solvates which can be administered by nebulization inhalation. The pharmaceutical formulations according to the invention meet high quality standards.

One aspect of the present invention is to provide an aqueous pharmaceutical formulation containing Olodaterol, or pharmaceutically acceptable salts or solvates thereof, which meets the high standards to achieve optimum nebulization of the formulation using the inhalers mentioned hereinbefore. Pharmaceutical formulations according to this aspect of the invention exhibit stability of several years, preferably at least 6 months, more preferably at least one year, most preferably at least three years.

Another aspect of the invention is to provide propellant-free formulations as solutions containing Olodaterol, or pharmaceutically acceptable salts or solvates thereof, which can be nebulized under pressure using an inhaler, which preferably is a nebulization inhaler device, to provide an aerosol, wherein the particle size of the aerosol falls reproducibly within a specified range.

Another aspect of the invention is to provide pharmaceutical formulations that are nebulization solutions comprising Olodaterol, or pharmaceutically acceptable salts or solvates thereof, and other inactive excipients which can be administered by nebulization inhalation using ultra-sonic based or air pressure based nebulizers/inhalers. Pharmaceutical formulations according to this aspect of the invention exhibit stability of several years, preferably at least 6 months, more preferably at least one year, most preferably at least three years.

More specifically, another aspect is to provide a stable pharmaceutical formulation of aqueous solutions containing Olodaterol, or pharmaceutically acceptable salts or solvates thereof, and other excipients which can be administered by nebulization inhalation using ultrasonic, jet or mesh nebulizers. According to this aspect of the invention, the pharmaceutical formulation is characterized by long term stability. The pharmaceutical formulations are stable for at least about 6-24 months when stored at a temperature of from about 15° C. to about 25° C.

### DETAILED DESCRIPTION OF THE INVENTION

It is very important to increase the lung deposition of drugs delivered by inhalation. In order to achieve a better distribution of active substances in the lung, it is advantageous to administer a liquid formulation without propellant gases using suitable inhalers to.

Currently, traditional pMDI or DPI (dry powder inhalation) devices only deliver about 20-30% of a drug into the lung, resulting in a significant amount of drug being deposited on the mouth and throat which is then delivered to the stomach causing unwanted side effects and or secondary absorption through the oral digestive system.

Therefore, there is a need on the art to improve the drug delivery by inhalation by significantly increasing lung deposition.

The pharmaceutical formulations of the invention is a solution that is converted into an aerosol destined for the lungs in the nebulizer. The pharmaceutical solution is sprayed with the nebulizer by high pressure.

Nebulization devices suitable for use with the pharmaceutical formulations of the present invention are those in which an amount of less than about 8 milliliters of pharmaceutical solution can be nebulized in one puff, preferably less than about 2 milliliters, most preferably less than about 1 milliliter, so that the inhalable part of aerosol corresponds to a therapeutically effective quantity. The average particle size of the aerosol formed from one puff is less than about 15 microns, preferably less than about 10 microns.

The formulations must not contain any ingredients which might interact with the inhaler to affect the pharmaceutical quality of the solution or of the aerosol produced. In addition, active substances in pharmaceutical formulations are very stable when stored and can be administered directly.

Therefore, one aspect of the present invention is to provide an aqueous pharmaceutical formulation containing Olodaterol, or pharmaceutically acceptable salts or solvates thereof, which meet the high standards needed in order to be able to achieve optimum nebulization of the solution using the inhalers mentioned hereinbefore. Preferably the active substance in the pharmaceutical formulation is stable, and the formulation has a storage time of some years, preferably at least one year, more preferably at least three years.

Another aspect of the current invention is to provide propellant-free formulations that are solutions containing Olodaterol, or pharmaceutically acceptable salts or solvates thereof, which are nebulized under pressure using an inhaler, preferably a nebulization inhaler, wherein the pharmaceutical composition delivered by the produced aerosol falls reproducibly within a specified range for particle size.

Another aspect is to provide an aqueous pharmaceutical formulation that is a solution containing Olodaterol, or pharmaceutically acceptable salts or solvates thereof, and other inactive excipients which can be administered by inhalation.

According to the invention, any pharmaceutically acceptable salts or solvates of Olodaterol may be used for the formulation. When the term Olodaterol is used within the scope of the present invention, it is to be taken as a reference to Olodaterol or pharmaceutically acceptable salts or solvates thereof, respectively.

In one embodiment, the salt of Olodaterol is Olodaterol hydrochloride.

In one embodiment, the active substance is Olodaterol hydrochloride.

In the formulations according to the invention, Olodaterol is dissolved in a solvent. In one embodiment, the solvent comprises water. In one embodiment, the solvent is water.

The concentration of Olodaterol in the finished pharmaceutical formulation depends on the therapeutic effects desired. In one embodiment, the concentration of Olodaterol is between about 18.2 mcg/100 ml and about 182 mg/100 ml, preferably between about 182 mcg/100 ml and about 1.82 mg/100 ml.

In the formulations according to the invention, if desired, the pH can be adjusted by adding a pH adjusting agent, such as an acid or a base. In one embodiment, citric acid and/or its salts thereof is added as the pH adjusting agent.

Other comparable pH adjusting agents can be used in the present invention. An example of a suitable pH adjusting agent is hydrochloric acid and/or sodium hydroxide.

The pH is selected to maintain stability of the active ingredients. In one embodiment, the pH ranges from about 2.0 to about 6.0, for example from about 2.8 to about 4.3.

In the formulations according to the invention, if desired, a stabilizer or complexing agent can be included in the formulation. Suitable stabilizers or complexing agents include, but are not limited to, edetic acid (EDTA) or one of the known salts thereof, disodium edetate or edetate disodium dihydrate. In one embodiment, the formulation contains edetic acid and/or a salt thereof.

Other comparable stabilizers or complexing agents can be used in the present invention. Other stabilizers or complexing agents include, but are not limited to, citric acid, edetate disodium, and edetate disodium dihydrate.

The phrase “complexing agent,” as used herein, means a molecule which is capable of entering into complex bonds. In one embodiment, these compounds have the effect of complexing cations. In one embodiment, the concentration of the stabilizer or complexing agent is from about 1 mg/100 ml to about 500 mg/100 ml, for example, from about 10 mg/100 ml to about 200 mg/100 ml. In one embodiment, the complexing agent is edetate disodium dihydrate in a concentration of from about 1 mg/100 ml to about 500 mg/100 ml.

In the formulations according to the invention, if desired, the isosmotic status of the formulation can be adjusted by adding an isosmotic adjusting agent, such as sodium chloride. In one embodiment, the formulation contains sodium chloride.

In one embodiment, the quantity of sodium chloride is from about 0.8% (w/w) to about 1.0% (w/w), for example, about 0.9% (w/w).

In one embodiment, if desired, a preservative may be added to the formulations according to the invention. In one embodiment, suitable preservatives include, but are not limited to, benzalkonium chloride, benzoic acid, sodium benzoate, and combinations thereof.

In one embodiment, a dose of Olodaterol hydrochloride includes from about 3 μg to about 80 preferably a dose of Olodaterol hydrochloride includes from about 3 μg to about 50 more preferably a dose of Olodaterol hydrochloride includes from about 5 μg to about 30 μg.

In one embodiment, Olodaterol is present in solution. It is advantageous if all the ingredients of the formulation are present in solution.

The phrase “additives,” as sued herein, means any pharmacologically acceptable and therapeutically useful substance which is not an active substance, but can be formulated together with the active substances in a pharmacologically suitable solvent, in order to improve the qualities of the formulation. Preferably, these substances have no appreciable pharmacological effects or, at least, no undesirable pharmacological effects in the context of the desired therapy.

Suitable additives include, but are not limited to, other stabilizers, complexing agents, antioxidants, surfactants, and/or preservatives which prolong the shelf life of the finished pharmaceutical formulation, vitamins, and/or other additives known in the art.

The pharmaceutical formulation is converted in the nebulizer into an aerosol that is destined for the lungs. The pharmaceutical solution is sprayed with the nebulizer by high pressure.

### EXAMPLES

Materials and Reagents:
Olodaterol hydrochloride, from Kalulan Science & Technology Co., Ltd. in Shanghai, China
Sodium chloride, from Merck
Citric acid, from Merck
Sodium hydroxide, from Titan Reagents Co., Ltd. in Shanghai, China
Hydrochloric acid, from Titan Reagents Co., Ltd. in Shanghai, China
50% benzalkonium chloride (referred to as BAC) aqueous solution is commercially available and may be purchased from Spectrum Pharmaceuticals Inc.
Edetate disodium dehydrate is commercially available and may be purchased from purchased from Merck & Co.

### Example 1

The preparation of sample I, sample II, and sample III inhalation solutions is as follows: active and inactive ingredients according to the amounts provided in Tables 1 and 2, were dissolved in 90 ml of purified water, and the resulting solution was then adjusted to the target pH with hydrochloric acid or citric acid or sodium hydroxide. Purified water was then added to final volume of 100 ml.

TABLE 1
Contents of Sample I of 100 ml of an Inhalable Formulation

| Ingredient | Sample I | |
|---|---|---|
| Olodaterol hydrochloride | 18.2 | mcg |
| Sodium chloride | 0.8 | g |

| | |
|---|---|
| hydrochloric acid | To pH 2.0 |
| Purified water | Added to 100 ml |

TABLE 2
Contents of Sample II and Sample III
of 100 ml of an Inhalable Formulation

| Ingredient | Sample II | | Sample III | |
|---|---|---|---|---|
| Olodaterol hydrochloride | 1.82 | mg | 182 | mg |
| Sodium chloride | 0.9 | g | 1.0 | g |

| | | |
|---|---|---|
| Citric acid or Sodium hydroxide | To pH 4.0 | To pH 6.0 |
| Purified water | Added to 100 ml | Added to 100 ml |

### Example 2

The preparation of sample IV inhalation solution is as follows: active and inactive ingredients according to the amounts provided in Table 3, were dissolved in 90 ml of purified water, and the solution was then adjusted to the target pH with citric acid or sodium hydroxide. Purified water was then added to final volume of 100 ml.

TABLE 3
Contents of Sample IV of 100 ml of an Inhalable Formulation

| Ingredient | Sample IV | |
|---|---|---|
| Olodaterol hydrochloride | 0.547 | mg |
| Sodium chloride | 0.9 | g |

| | |
|---|---|
| citric acid or Sodium hydroxide | To pH 3.9 |
| Purified water | Added to 100 ml |

### Example 3

TABLE 4
Osmotic Pressure of Sample IV

| Sample Number | Osmotic pressure |
|---|---|
| Sample IV | 287 mOsm |

### Example 4

Influence of pH on Stability:

Stability is highly dependent on pH. Eight samples were prepared according to Table 5. Olodaterol hydrochloride (referred to as OH) in the amounts provided in Table 5 was dissolved in 90 ml of purified water. The pH of samples 1-7 were then adjusted with HCl to a pH of 2.8, 3.1, 3.4, 3.7, 4.0, 4.3 and 4.6, respectively. The pH of sample 8 pH was unadjusted. The resulting mixtures were then sonicated until the components completely dissolved. Purified water was then added to a final volume of 100 ml for each sample.

TABLE 5
Formulation Design of OH Screening at Different pH Values

| Ingredients | Sample1 | Sample2 | Sample3 | Sample4 | Sample5 | Sample6 | Sample7 | Sample8 |
|---|---|---|---|---|---|---|---|---|
| OH | 25 mg | 25 mg | 25 mg | 25 mg | 25 mg | 25 mg | 25 mg | 25 mg |
| HCl | Adjust to | Adjust to | Adjust to | Adjust to | Adjust to | Adjust to | Adjust to | pH not |
| | pH 2.8 | pH 3.1 | pH 3.4 | pH 3.7 | pH 4.0 | pH 4.3 | pH 4.6 | adjusted |
| | | | | | | | | (about pH |
| | | | | | | | | 6) |
| Purified | Added to | Added to | Added to | Added to | Added to | Added to | Added to | Added to |
| water | 100 ml | 100 ml | 100 ml | 100 ml | 100 ml | 100 ml | 100 ml | 100 ml |

Method for Impurity Analysis:
Mobile phase A: Weigh 2.72 g KH₂PO₄, dissolve in 1 L water, add 2 mL Triethanolamine into solution before adjusting pH to 6.0 with H₃PO₄.
Mobile phase B: CH₃OH
Column: Inertsil ODS-3 (5.0 μm, 250×4.6 mm)
Column Temperature: 30° C.
Flow rate: 1.0 mL/min
Injection Volume: 20 μL
Run Time: 75 minutes
Detection Wavelength: 220 nm
Gradient elution:

| Time(min) | A(%) | B(%) |
|---|---|---|
| 0 | 90 | 10 |
| 15 | 70 | 30 |
| 20 | 62 | 38 |
| 30 | 52 | 48 |
| 50 | 30 | 70 |
| 65 | 30 | 70 |
| 66 | 90 | 10 |
| 75 | 90 | 10 |

The contents of the eight samples is provided in Table 5. Each sample was stored at 60° C. for 14 days. The stability profile of the samples is provided in Tables 6-8.

TABLE 6
Stability at Different pH Values (Conditions: 0 day)

| | Sample1 | Sample2 | Sample3 | Sample4 | Sample5 | Sample6 | Sample7 | Sample8 |
|---|---|---|---|---|---|---|---|---|
| RRT | % Area | % Area | % Area | % Area | % Area | % Area | % Area | % Area |
| 0.71 | 0.29 | 0.29 | 0.29 | 0.29 | 0.29 | 0.29 | 0.29 | 0.29 |
| 1.21 | 0.34 | 0.33 | 0.34 | 0.34 | 0.34 | 0.34 | 0.34 | 0.34 |
| 1.23 | 0.1 | 0.09 | 0.09 | 0.1 | 0.09 | 0.1 | 0.09 | 0.09 |
| 1.28 | 0.38 | 0.37 | 0.38 | 0.38 | 0.39 | 0.38 | 0.36 | 0.37 |
| Maximum | 0.38 | 0.37 | 0.38 | 0.38 | 0.39 | 0.38 | 0.36 | 0.37 |
| impurity | | | | | | | | |
| Total | 1.11 | 1.08 | 1.1 | 1.11 | 1.11 | 1.11 | 1.08 | 1.09 |

TABLE 7
Stability at Different pH Values (Conditions: 60° C. 7 day, 75% RH)

| | Sample1 | Sample2 | Sample3 | Sample4 | Sample5 | Sample6 | Sample7 | Sample8 |
|---|---|---|---|---|---|---|---|---|
| RRT | % Area | % Area | % Area | % Area | % Area | % Area | % Area | % Area |
| 0.12 | 0.12 | 0.1 | 0.09 | 0.07 | 0.1 | 0.1 | 0.1 | 0.06 |
| 0.70 | 0.38 | 0.37 | 0.34 | 0.32 | 0.35 | 0.35 | 0.38 | 0.46 |
| 1.21 | 0.3 | 0.3 | 0.26 | 0.27 | 0.26 | 0.26 | 0.26 | 0.25 |
| 1.27 | 0.31 | 0.32 | 0.32 | 0.36 | 0.3 | 0.35 | 0.33 | 0.33 |
| 1.50 | 0.09 | 0.1 | 0.12 | 0.14 | 0.17 | 0.18 | 0.21 | 0.3 |
| Maximum | 0.38 | 0.37 | 0.34 | 0.32 | 0.35 | 0.35 | 0.38 | 0.46 |
| impurity | | | | | | | | |
| Total | 1.08 | 1.09 | 1.04 | 1.09 | 1.08 | 1.14 | 1.18 | 1.34 |

TABLE 8
Stability at Different pH Values (Conditions: 60° C. 14 day, 75% RH)

| | | Sample1 | Sample2 | Sample3 | Sample4 | Sample5 | Sample6 | Sample7 | Sample8 |
|---|---|---|---|---|---|---|---|---|---|
| | RRT | % Area | % Area | % Area | % Area | % Area | % Area | % Area | % Area |
| Impurity 1 | 0.12 | 0.1 | 0.1 | 0.12 | 0.09 | 0.12 | 0.16 | 0.06 | 0.05 |
| Impurity 2 | 0.32 | 0.01 | 0.01 | 0.01 | — | 0.01 | 0.04 | 0.12 | 0.11 |
| Impurity 3 | 0.70 | 0.43 | 0.44 | 0.43 | 0.36 | 0.39 | 0.51 | 0.68 | 0.62 |
| Impurity 4 | 0.84 | — | — | — | — | — | — | 0.16 | 0.17 |
| Impurity 5 | 1.05 | 0.07 | 0.07 | 0.09 | 0.06 | 0.11 | 0.22 | 0.14 | 0.1 |
| Impurity 6 | 1.22 | 0.3 | 0.29 | 0.29 | 0.3 | 0.29 | 0.28 | 0.29 | 0.28 |
| Impurity 7 | 1.28 | 0.31 | 0.31 | 0.3 | 0.32 | 0.3 | 0.28 | 0.29 | 0.3 |
| Impurity 8 | 1.51 | 0.15 | 0.18 | 0.26 | 0.2 | 0.24 | 0.29 | 0.53 | 0.46 |
| Maximum impurity | | 0.43 | 0.44 | 0.43 | 0.36 | 0.39 | 0.51 | 0.68 | 0.62 |
| | Total | 1.37 | 1.4 | 1.5 | 1.33 | 1.46 | 1.78 | 2.27 | 2.09 |

As can be seen from Tables 6-8, the OH containing solution is stable at pH 2.8 to 6.0, the OH solution is most stable at pH 2.8 to 4.3.

### Example 5

Aerodynamic Particle Size Distribution:

50% benzalkonium chloride aqueous solution (referred to as 50% BAC), edetate disodium dihydrate, and citric acid (referred to as CA) according to the amounts provided in Table 9 were dissolved in 900 ml of purified water. The pH was then adjusted to pH 3.4 with HCl. OH according to the amount provided in Table 9 was then added to the solution, and the resulting mixture sonicated until the components were completely dissolved. Purified water was then added to a final volume of 1000 ml.

The formula of the sample is shown in Table 9.

TABLE 9
Contents of Sample 9

| Ingredients | Sample 9 | |
|---|---|---|
| OH | 0.25 | g |
| CA | 0.003 | g |
| 50% BAC | 0.2 | g |
| EDTA | 0.055 | g |
| HCl | Adjust to 3.4 | |
| Purified water | 1000 | ml |

The aerodynamic particle size distribution was determined using an Andersen Scale Impactor (ACI). The inhalation device, named Respimat, was purchased from Boehringer Ingelheim Co., Ltd. The Respimat inhaler was held close to the ACI inlet until no aerosol was visible. The flow rate of the ACI was set to 28.3 L/minute and was operated under ambient temperature and a relative humidity (RH) of 90%.

The solution of sample 9 was discharged into the ACI. Fractions of the dose were deposited at different stages of the ACI, in accordance with the particle size of the fraction. Each fraction was washed from the stage and analyzed using HPLC.

The results are provided in Table 10 below.

TABLE 10
Aerodynamic Particle Size Distribution of OH Inhalation
Formulation Sample 9 Administered by Respimat Inhalation

| | | | Cut-off |
|---|---|---|---|
| | Dosage | Percentage content | diameter |
| Deposited | mcg | at all levels | (μm) |
| Throat | 0.5533 | 17.81% | |
| Stage 0 | 0.2471 | 7.96% | 9.0 |
| Stage 1 | 0.4189 | 13.49% | 5.8 |
| Stage 2 | 0.3521 | 11.34% | 4.7 |
| Stage 3 | 0.4891 | 15.75% | 3.3 |
| Stage 4 | 0.3073 | 9.89% | 2.1 |
| Stage 5 | 0.0461 | 1.48% | 1.1 |
| Stage 6 | 0.0925 | 2.98% | 0.7 |
| Stage 7 | 0.2385 | 7.68% | 0.4 |
| Stage F | 0.3613 | 11.63% | |
| Theoretical dose(μg) | 2.7625 | | |
| Actual test dose(μg) | 3.1062 | | |
| Recovery rate % | 112.44% | | |
| Fine Particle Fraction (FPF) | 60.75% | | |

The larger the FPF value, the higher the atomization efficiency.

The above results confirmed that the formulation of the present invention has a good atomization effect.

### Example 6

Stability Experiment:

50% benzalkonium chloride aqueous solution (referred to 50% BAC), edetate disodium dihydrate, and citric acid (referred to CA) according to the amounts provided in Table 11 were dissolved in 180 ml of purified water. The pH was then adjusted with HCl to pH 3.1, 3.4, 3.7, 3.85, respectively. OH according to the amounts provided in Table 11 was added to each solution and the resulting mixtures sonicated until the components were completely dissolved. Purified water was then added to a final volume of 200 ml for each sample.

TABLE 11
Contents of Sample 10-13

| Ingredients | Sample 10 | | Sample 11 | | Sample 12 | | Sample 13 | |
|---|---|---|---|---|---|---|---|---|
| OH | 50 | mg | 50 | mg | 50 | mg | 50 | mg |
| 50% BAC | 40 | mg | 40 | mg | 40 | mg | 40 | mg |
| EDTA | 22 | mg | 22 | mg | 22 | mg | 22 | mg |
| CA | 6 | mg | 6 | mg | 6 | mg | 6 | mg |
| HCl | 3.1 | | 3.4 | | 3.7 | | 3.85 | |
| Purified | 200 | ml | 200 | ml | 200 | ml | 200 | ml |
| water | | | | | | | | |

Sample 10-13 were maintained at 40° C./75% RH for 0, 1, 2, 3, 6 months.

Impurity Analysis Method:
Buffer: Weigh 2.72 g KH2PO4, dissolve in 1 L water, add 2 mL Triethanolamine into solution before adjusting pH to 6.0 with H₃PO₄.
Mobile phase A: buffer:CH₃OH=9:1
Mobile phase B: buffer:CH₃OH=3:7
Column: YMC-Triart C18 (5.0 μm, 250×4.6 mm)
Column Temperature: 30° C.
Flow rate: 1.0 mL/min
Injection Volume: 30 μL
Run Time: 100 minutes
Detection Wavelength: 220 nm
Gradient elution:

| Time(min) | A(%) | B(%) |
|---|---|---|
| 0 | 100 | 10 |
| 15 | 84 | 16 |
| 35 | 67 | 33 |
| 45 | 50 | 50 |
| 55 | 42 | 58 |
| 75 | 0 | 100 |
| 90 | 0 | 100 |
| 91 | 100 | 0 |
| 100 | 100 | 0 |

The stability profile is provided below in Tables 12-17.

TABLE 12
Stability Profile of Samples 10-13 (Conditions: 40°
C. ± 2° C./75% ± 5% RH)

| | | percentage (%) | | |
|---|---|---|---|---|
| | Sample | OH | 50% BAC | EDTA |
| 0 Month | Sample 10 | 101.2 | 101.95 | 99.09 |
| | Sample 11 | 101.64 | 100.25 | 99.82 |
| | Sample 12 | 102.12 | 95.20 | 101.55 |
| | Sample 13 | 100.56 | 99.35 | 102.09 |
| 1 Month | Sample 10 | 101.44 | 98.55 | 99.45 |
| | Sample 11 | 102.56 | 101.40 | 100.91 |
| | Sample 12 | 102.52 | 99.90 | 102.09 |
| | Sample 13 | 102.56 | 94.85 | 100.36 |
| 2 Months | Sample 10 | 100.92 | 99.25 | 98.91 |
| | Sample 11 | 100.72 | 98.40 | 99.82 |
| | Sample 12 | 100.52 | 94.20 | 101.55 |
| | Sample 13 | 100.52 | 97.10 | 99.55 |
| 3 Months | Sample 10 | 102.46 | 92.30 | 98.73 |
| | Sample 11 | 101.39 | 90.80 | 98.82 |
| | Sample 12 | 100.05 | 86.45 | 99.45 |
| | Sample 13 | 102.07 | 89.30 | 99.91 |
| 6 Months | Sample 10 | 99.44 | 99.25 | 97.73 |
| | Sample 11 | 99.52 | 97.85 | 98.00 |
| | Sample 12 | 100.28 | 93.45 | 99.27 |
| | Sample 13 | 99.36 | 96.35 | 97.73 |

TABLE 13
The Stability Profile of Samples 10-13 at 0 days (Conditions: 40°
C. ± 2° C./75% ± 5% RH)

| | | | Sample | Sample | Sample | Sample |
|---|---|---|---|---|---|---|
| | | | 10 | 11 | 12 | 13 |
| | | 0 Day | 0 Day | 0 Day | 0 Day | 0 Day |
| | RT | RRT | % Area | % Area | % Area | % Area |
| Unknown | 33.76 | 0.64 | 0.31 | 0.31 | 0.31 | 0.33 |
| impurity | | | | | | |
| Unknown | 61.10 | 1.17 | 0.44 | 0.44 | 0.47 | 0.47 |
| impurity | | | | | | |
| Unknown | 63.42 | 1.21 | 0.37 | 0.37 | 0.39 | 0.39 |
| impurity | | | | | | |
| Unknown | 71.27 | 1.35 | 0.14 | 0.16 | 0.16 | 0.17 |
| impurity | | | | | | |
| | Total | | 1.26 | 1.28 | 1.33 | 1.36 |

TABLE 14
The Stability Profile of Samples 10-13 at 1 month (Conditions:
40° C. ± 2° C./75% ± 5% RH)

| | | | Sample | Sample | Sample | Sample |
|---|---|---|---|---|---|---|
| | | | 10 | 11 | 12 | 13 |
| Time | | | 1 Month | | | |
| name | RT | RRT | % Area | % Area | % Area | % Area |
| Unknown | 33.88 | 0.64 | 0.32 | 0.33 | 0.31 | 0.31 |
| impurity | | | | | | |
| Unknown | 62.47 | 1.17 | 0.44 | 0.44 | 0.44 | 0.44 |
| impurity | | | | | | |
| Unknown | 64.76 | 1.21 | 0.3 | 0.3 | 0.29 | 0.3 |
| impurity | | | | | | |
| Unknown | 72.48 | 1.35 | 0.14 | 0.2 | 0.17 | 0.19 |
| impurity | | | | | | |
| | Total | | 1.2 | 1.27 | 1.21 | 1.24 |

TABLE 15
The Stability Profile of Sample 10-13 at 2 months (Conditions:
40° C. ± 2° C./75% ± 5% RH)

| | | | Sample | Sample | Sample | Sample |
|---|---|---|---|---|---|---|
| | | | 10 | 11 | 12 | 13 |
| Time | | | 2 Months | | | |
| name | RT | RRT | % Area | % Area | % Area | % Area |
| Unknown | 33.32 | 0.64 | 0.34 | 0.36 | 0.33 | 0.35 |
| impurity | | | | | | |
| Unknown | 62.11 | 1.17 | 0.4 | 0.39 | 0.4 | 0.4 |
| impurity | | | | | | |
| Unknown | 64.43 | 1.21 | 0.3 | 0.29 | 0.3 | 0.29 |
| impurity | | | | | | |
| Unknown | 72.30 | 1.35 | 0.2 | 0.25 | 0.3 | 0.3 |
| impurity | | | | | | |
| | Total | | 1.24 | 1.29 | 1.33 | 1.34 |

TABLE 16
The Stability Profile of Sample 10-13 at 3 months (Conditions:
40° C. ± 2° C./75% ± 5% RH)

| | | | Sample | Sample | Sample | Sample |
|---|---|---|---|---|---|---|
| | | | 10 | 11 | 12 | 13 |
| Time | | | 3 Months | | | |
| name | RT | RRT | % Area | % Area | % Area | % Area |
| Unknown | 33.05 | 0.64 | 0.32 | 0.35 | 0.32 | 0.33 |
| impurity | | | | | | |
| Unknown | 62.33 | 1.17 | 0.36 | 0.37 | 0.37 | 0.38 |
| impurity | | | | | | |
| Unknown | 64.58 | 1.21 | 0.29 | 0.29 | 0.29 | 0.29 |
| impurity | | | | | | |
| Unknown | 72.31 | 1.35 | 0.23 | 0.26 | 0.29 | 0.33 |
| impurity | | | | | | |
| | Total | | 1.2 | 1.27 | 1.27 | 1.33 |

TABLE 17
The Stability Profile of Sample 10-13 at 6 months (Conditions:
40° C. ± 2° C./75% ± 5% RH)

| | | | Sample | Sample | Sample | Sample |
|---|---|---|---|---|---|---|
| | | | 10 | 11 | 12 | 13 |
| Time | | | 6 Months | | | |
| name | RT | RRT | % Area | % Area | % Area | % Area |
| Unknown | 35.13 | 0.64 | 0.34 | 0.35 | 0.32 | 0.33 |
| impurity | | | | | | |
| Unknown | 63.679 | 1.17 | 0.39 | 0.4 | 0.4 | 0.39 |
| impurity | | | | | | |
| Unknown | 65.91 | 1.21 | 0.28 | 0.27 | 0.28 | 0.29 |
| impurity | | | | | | |
| Unknown | 73.636 | 1.35 | 0.28 | 0.54 | 0.67 | 0.68 |
| impurity | | | | | | |
| | Total | | 1.29 | 1.56 | 1.67 | 1.69 |

As shown in Tables 12-17, at pH 3.1-3.85 the OH solutions showed good stability. OH solutions ranging from a pH of about 3.1 to about 3.85 were stable for about 6 months at 40° C.±2° C./75%±5% RH.

While various embodiments of the present invention have been described above, it should be understood that they have been presented by way of example only, and not limitation. For example, the present invention is not limited to the physical arrangements or dimensions illustrated or described. Nor is the present invention limited to any particular design or materials of construction. As such, the breadth and scope of the present invention should not be limited to any of the above-described exemplary embodiments, but should be defined only in accordance with the following claims and their equivalents.

What is claimed is:

1. A propellant-free inhalation formulation comprising Olodaterol, or a pharmaceutically acceptable salt thereof, a pH adjusting agent, optionally including a pharmacologically acceptable additive, dissolved in a solvent.

2. The formulation of claim 1, wherein the pharmaceutically acceptable salt is Olodaterol hydrochloride.

3. The formulation of claim 2, wherein Olodaterol hydrochloride is present in an amount ranging from about 182 mcg/100 ml to about 182 mg/100 ml.

4. The formulation of claim 1, comprising a pharmacologically acceptable additive, wherein the pharmacologically acceptable additive is an isosmotic adjusting agent selected from the group consisting of sodium chloride, glucose, mannitol, glucitol, and mixtures thereof.

5. The formulation of claim 4, wherein the isosmotic adjusting agent is present in an amount ranting from about 0.8% (w/w) to about 1% (w/w).

6. The formulation of claim 1, wherein the solvent is water.

7. The formulation of claim 1, wherein the pH adjusting agent is selected from the group consisting of citric acid-citrate, citric acid, hydrochloric acid, and sodium hydroxide.

8. The formulation of claim 1, wherein the formulation has a pH ranging from about 1.0 to about 6.0.

9. The formulation of claim 1, wherein the formulation has a pH ranging from about 2.8 to about 4.3.

10. The formulation of claim 1, wherein the pharmacologically acceptable additive is selected from the group consisting of edetic acid, edetate disodium dehydrate, edetate disodium, citric acid, and combinations thereof.

11. The formulation of claim 1, comprising a pharmacologically acceptable additive, wherein the pharmacologically acceptable additive is selected from the group consisting of benzalkonium chloride, benzoic acid, sodium benzoate, and combinations thereof.

12. The formulation of claim 1, comprising a pharmacologically acceptable additive, wherein the pharmacologically acceptable additive is present in an amount ranging from about 1 mg/100 ml to about 500 mg/100 ml.

13. The formulation according to claim 1, wherein the formulation has osmotic pressure ranging from about 100 mOsm to about 400 mOsm.

14. A method of treating asthma or COPD in a patient, comprising administering to the patient a therapeutically effective amount of the pharmaceutical preparation according to claim 1 by inhalation.

15. The method of claim 14, wherein the pharmaceutically acceptable salt is Olodaterol hydrochloride, and the Olodaterol hydrochloride is administered at a dose ranging from about 3 μg to about 80 μg.

16. The method of claim 15, wherein the Olodaterol hydrochloride is administered at a dose ranging from about 5 μg to about 30 μg.

17. The method of claim 14, wherein the pharmaceutical formulation is administered using a nebulization inhalation device to provide an inhalable aerosol of the pharmaceutical formulation.

18. The method of claim 16, wherein the inhalable aerosol has a D50 that is less than about 10 μm.

19. The method of claim 17, wherein the inhalable aerosol has an average particle size of less than about 15 microns.
